(19) United States
(12) Patent Application Publication
(10) Pub. No.: US 2024/0254591 A1
(43) Pub. Date: Aug. 1, 2024

(54) PROCESSES AND METHODS FOR BIO-EXTRACTION OF TRACE METALS FROM METAL-OXIDE CONTAINING MATERIALS

(71) Applicant: Impossible Mining Inc., San Jose, CA (US)

(72) Inventors: Oliver Gunasekara, San Jose, CA (US); Kenneth Nealson, San Jose, CA (US); Thomas Ishoey, San Jose, CA (US); Luis Bernal, San Jose, CA (US); Roman Barco, San Jose, CA (US)

(21) Appl. No.: 18/560,633

(22) PCT Filed: May 12, 2022
(86) PCT No.: PCT/US22/72282

§ 371 Date: Nov. 13, 2023

Related U.S. Application Data
(60) Provisional application No. 63/187,748, filed on May 12, 2021.

(51) Int. Cl. C22B 3/18; C12P 3/00; C21B 13/00; C22B 1/24; C22B 47/00

(52) U.S. Cl. CPC C22B 3/18; C12P 3/00; C21B 13/008; C22B 1/24; C22B 47/00

(57) ABSTRACT

Provided herein are processes and methods for the bio-extraction of trace metals from a metal-oxide containing starting material (e.g., a metallic ore). The processes and methods may utilize metal-oxide reducing bacteria to electrochemically reduce metal oxides in the ores, thereby freeing valuable trace metals. In preferred embodiments, the processes and methods may utilize metal-oxide reducing bacteria from the group Shewanellaceae and/or the group Geobacteraceae.

### CROSS REFERENCE TO RELATED APPLICATIONS

This application claims priority to U.S. Provisional Application No. 63/187,748, filed May 12, 2021, which is incorporated herein by reference in its entirety.

### FIELD

Provided herein are processes and methods for the extraction of trace metals from a metal-bearing starting material which directly, or in combination with other processes detailed below, use metal-reducing bacteria to solubilize metals.

### BACKGROUND

The world, and especially the United States, lacks a reliable supply of critical minerals that can be made into metals for everything from electric vehicle (EV) batteries to solar panels to wind turbines. Reserves of many trace metals, including nickel, copper and cobalt, are already becoming depleted, and are expensive to recover. Ores available today contain much less of these metals than in decades past, meaning they are less efficient to process and generate higher volumes of waste. Furthermore, the energy and environmental impact of current mining technology in the extraction of metals from ores using traditional processing is unsustainable and does not meet the global objectives embodied in the UN sustainable development goals.

Current methods of extracting metals from ores include acid leaching, bio leaching, and (more frequently) a combination of these two methods. Both acid leaching and bio leaching are costly, time consuming processes that generate very large levels of toxic acidic waste materials. For example, High Pressure Acid Leaching (HPAL) is currently the dominant method of extracting nickel from laterite ore. This method requires large amounts of energy, emits massive amounts of carbon, and generates huge amounts of acidic slurry waste, often leading to environmental disasters.

It is therefore desirable to develop new methods for extraction of trace metals from ores that are economical and environmentally sustainable. In particular, it is desirable to provide a low energy pathway for processing ore that has a low carbon footprint and a lower environmental impact than currently used methods.

### SUMMARY

In one aspect, provided herein is a method for the extraction of a trace metal from a granular material comprising a metal oxide, the method comprising contacting the granular material with at least one species of metal-oxide reducing bacteria in an aqueous medium, thereby (1) converting at least a portion of the metal oxide to a water-soluble metal salt, and (2) releasing at least a portion of the trace metal into the aqueous medium; and recovering at least a portion of the trace metal from the aqueous medium.

In a further aspect, provided herein is a method for the extraction of a trace metal from a granular material comprising a metal sulfide, the method comprising (1) contacting the granular material with at least one species of bacteria selected from the group consisting of neutrophilic metal-oxidizing bacteria and sulfur-oxidizing bacteria in an aqueous medium, thereby converting at least a portion of said metal sulfide to a metal oxide; (2) subsequently contacting the granular material with at least one species of metal-oxide reducing bacteria in an aqueous medium, thereby (a) converting at least a portion of the metal sulfide to a water soluble metal salt, and (b) releasing at least a portion of the trace metal into the aqueous medium; and (3) recovering at least a portion of the trace metal from the aqueous medium.

In a further aspect, provided herein is a method for comminuting a starting material comprising a metal oxide, the method comprising at least partially submerging the starting material in an aqueous medium comprising at least one species of metal oxide reducing bacteria; and mechanically crushing or vibrating the starting material; thereby producing a granular material having a smaller mean particle size than the starting material.

Also provided herein is a trace metal obtained using a method as described herein. Non-limiting examples of trace metals that may be recovered using the methods provided herein include lithium, zinc, copper, chromium, nickel, cobalt, vanadium, molybdenum, cadmium, rare earth elements, and platinum group elements.

Other objects and features will be in part apparent and in part pointed out hereinafter.

### DESCRIPTION OF THE DRAWINGS

FIG. 1 is a schematic diagram of an optional preprocessing stage as provided herein. The preprocessing stage may comprise, as a non-limiting example, one or more of the following steps: comminution of the starting material; oxidation of the starting material; and softening of the starting material. Each of the alternatives depicted in FIG. 1 (e.g., alternatives A, B, C, D, E, F, and G) is within the scope of the present disclosure.

FIG. 2 is a general process flow diagram that depicts an exemplary process for the bio-extraction of trace metals from a starting material comprising a metal oxide. The exemplary process includes: (1) a preprocessing stage, where the starting material is “softened” by pretreatment and/or crushed or ground to form a granular material and/or oxidized; (2) a biomass generation stage, wherein a population or consortium of metal oxide-reducing bacteria is maintained; (3) a bio-extraction stage, where the granular material is contacted with at least one species of metal oxide-reducing bacteria; (4) a separation stage where metal oxide reducing bacteria are separated from dissolved trace metals; and (5) a recovery stage wherein the desirable trace metals are separated and recovered; and (6) a waste water recovery stage wherein spent medium is recycled.

### DETAILED DESCRIPTION

Provided herein are processes and methods for the bio-extraction of trace metals from a metal containing material, which is preferably a metal-oxide containing starting material (e.g., metallic ores, polymetallic nodules, or recycled waste material). As discussed in further detail below, the processes and methods may utilize metal-oxide reducing bacteria to electrochemically reduce metal oxides in the ores, thereby freeing valuable trace metals.

These processes and methods are generally referred to herein as “bio-extraction” processes and methods. As explained in further detail below, bio-extraction utilizes bacteria that actively respire the metal oxides in ore, electrochemically reducing the metal oxides to soluble salts, and releasing trace metals bound within the ore. The bacteria may be fed an inexpensive substrate (e.g., sodium lactate) from which the bacteria extract electrons, and the bacteria then use the electrons to actively reduce the metal oxides present in the ore. When the metal oxides are solubilized, the bound trace metals are released into solution, and can be further separated and purified by standard industrial methods known to those skilled in the art. The waste products are minimal, typically consisting of bacterial biomass (which may be used to inoculate future batches), and iron carbonates or phosphates of both iron and manganese (all of which are valuable commodities).

Many different species of bacteria are capable of being utilized in the bio-extraction processes and methods provided herein, including: (1) marine and freshwater forms; (2) low to high temperature tolerant forms; (3) bacteria that utilize different energy and/or carbon sources; and (4) bacteria that have metabolic preferences for particular iron or manganese oxides. In one aspect, therefore, the present processes and methods comprise the selection of an ore-specific consortium of bacteria that is optimized for bio-extraction of trace metals from a particular source. It is also possible to construct a consortium of bacteria that is optimized for operation over a specified range of temperatures, and/or in seawater, obviating the need for heating the samples or for using large amounts of freshwater.

The bio-extraction processes described herein are significantly different from the bio-leaching processes currently used in the art. A conventional bio-leaching process uses bacteria to generate strong acid, which in turn is used to leach the trace metals from the metal oxides present in the ore. In contrast, the bio-extraction process provided herein does not rely upon the generation or use of a strong acid, and in fact can be conducted at neutral pH (e.g., a pH from about 6 to about 8). Instead, the bio-extraction process utilizes metal-oxide reducing bacteria that directly solubilize the metal oxides, thereby releasing the bound trace metals. This reaction occurs faster than in acid leaching, as it is a direct metabolic reaction of electron transfer from the bacteria to the metal oxide. Additionally, the bio-extraction process utilizes bacteria that are motile, greatly reducing the stagnant boundary layers and passivation involved with diffusion-limited bio-leaching processes.

In addition, the bio-extraction technology provided herein may be applied to heap or pile leaching processes that typically operate on particles having an average particle size of ¼″ to ¾″ (about 0.5 cm to about 2 cm). Currently, in such processes the starting material (e.g., ore) is moistened with solutions loaded with sulfuric acid and with oxidizing bacteria that dissolve metals in a thin layer contact process. The bio-extraction process provided herein may advantageously comprise using a neutral solution loaded with bio-extraction bacteria that can dissolve the metal oxides and free the bound trace metals, and then continue the metal separation and recirculation of spent solution.

Embodiments of the processes and methods described herein may provide one or more advantages relative to prior art processes. For example, the processes and methods described herein may be carried out at neutral pH and do not require the production of environmentally unfriendly strong acid waste. Additionally, the processes and methods described herein may be carried out using seawater or brackish water, and do not require a source of freshwater. These and other potential advantages are described in further detail below.

### Metal-Oxide Reducing Bacteria

The bio-extraction processes and methods described herein utilize one or more species of metal-oxide reducing bacteria.

Non-limiting examples of metal-oxide reducing bacteria include the group Shewanellaceae and the group Geobacteraceae. For example, bacteria in the group Shewanellaceae have the ability, via a process called extracellular electron transport (“EET”), to electrochemically reduce insoluble iron and manganese oxides. Shewanellaceae utilize oxidized iron and manganese compounds for cellular respiration (in the absence of oxygen) and, in the process, convert the insoluble oxides to soluble salts (e.g., FeCl₂ or MnCl₂). Bacteria in the group Geobacteraceae, while metabolically much different from the Shewanellaceae, also possess similar metal-oxide reducing abilities. Accordingly, in a preferred embodiment, for each starting material (e.g., ore) to be extracted, the bacterial inoculum comprises at least one species selected from Shewanella spp., or at least one species selected from Geobacter spp., or a combination thereof.

In preferred embodiments, the process or method will utilize two or more species of metal-oxide reducing bacteria. The various species and strains of Shewanella and Geobacter have individual abilities with regard to which metal oxides (iron or manganese), even among the manganese or iron oxides, which particular crystalline forms they prefer. In general, the Shewanella spp. are to be more suited to reduction of manganese oxides, while fitting with their ecological location at oxic/anoxic interfaces. Conversely, the Geobacter spp. are, in general, more suited for the reduction of iron oxides. Accordingly, when the starting material comprises a combination of iron oxides and manganese oxides, using two or more species of metal-oxide reducing bacteria selected from the Shewanella spp. and Geobacter spp. is preferred. For example, in some embodiments, the one or more species of metal-oxide reducing bacteria comprise at least one species in the group Shewanella and at least one species in the group Geobacter.

For illustrative purposes, an exemplary embodiment of the process utilizing multiple strains of bacteria may proceed as follows:
1. Strains of bacteria that are known to effectively reduce metal oxides are grown in a nutrient medium to a cell density of 10⁹ to 10¹⁰ cells per ml (phase 2). Each strain to be used in a consortium is grown separately, in the optimal medium for that strain.
2. Bacteria are added to the sample to be bio-extracted at least at a concentration of 10⁷ cells/ml, for each member of the consortium (i.e., a consortium of 4 strains will contain a concentration of at least 4×10⁷ total bacteria). The substrate for the bacteria is added at a concentration appropriate for complete dissolution of the iron and/or manganese oxides present in the sample.
3. The bio-extraction reactor is sealed to allow oxygen consumption by the bacteria, and monitored for the production of reduced metals (Fe(II) and/or Mn(II) (phase 3).
4. When the increase in soluble reduced metals ceases, reaction will be stopped, and the slurry moved to the separation phase (phase 4).

In addition, even among bacterial species or strains within the same group, the ability to electrochemically reduce particular species of iron oxide or manganese oxide may vary significantly. For example, in the group Shewanellaceae, different isolates (species or strains) have preferences for different metal oxides (e.g., iron vs manganese). In some cases, individual strains have preferences for different iron oxides (e.g., FeOOH, Fe(OH)₃, or Fe₂O₃) or between different mineral forms of manganese oxides (e.g., j-MnO₂, birnessite, or pyrolusite).

In preferred embodiments, selection of the one or more species of metal-oxide reducing bacteria will therefore be based upon the following parameters: (1) the temperature range within the reaction vessel (which typically ranges from about 4° C. to about 35° C.); (2) salinity requirements and/or tolerances; (3) ability to reduce metal oxides present in the starting material (for example, manganese oxides or iron oxides); (4) the carbon source(s) capable of being used for growth; (5) sensitivity to oxygen (obligate vs facultative anaerobes); (6) tolerance to high levels of soluble Mn and/or Fe; (7) tolerance to the trace metals released during the bio-extraction process; and (8) the pH range within the reaction vessel (which typically ranges from a pH of from about 6 to about 8.5).

Representative, non-limiting examples of suitable Shewanella species and strains are provided in Table 1 below. As shown in the table, the listed species and strains generally overlap with respect to acceptable conditions of temperature, salinity, and carbon source, and are therefore compatible for growth as mixed cultures. All Shewanella species and strains listed in Table 1 are capable of reduction of both iron and manganese. Additionally, all listed Shewanella species and strains are facultative anaerobes, capable of respiration of oxygen. Under anoxic conditions, all listed strains are capable of lactate utilization, while none of them can utilize acetate.

TABLE 1

| Bacterial | T | | | Carbon |
|---|---|---|---|---|
| Strain(s) | (° C.) | Salinity* | Metals | utilized |
| S. oneidensis MR-1 | 10-30 | L-M | Fe/Mn/S | lactate |
| S. amazonensis SB2B | 10-30 | L-H | Fe/Mn/S | lactate |
| S. baltica OS185 | 4-20 | M-H | Fe/Mn | lactate |
| S. putrefaciens CN-32 | 10-30 | L-H | Fe/Mn/S | lactate |
| S. loihica PV-4 | 4-20 | M-H | Fe/Mn | lactate |
| S. sp. MR-4 | 10-30 | L-H | Fe/Mn | lactate |
| S. sp. MR-7 | 10-30 | L-H | Fe/Mn | lactate |
| S. sp. W3-18-1 | 4-30 | M-H | Fe/Mn | lactate |
| S. sp. ANA-3 | 10-30 | M-H | Fe/Mn | lactate |
| S. violaceae DSS12 | 4-30 | M-H | Fe/Mn | lactate |

*Salinity is indicated as follows: L = low; M = medium; H = high

Non-limiting examples of particularly preferred Shewanellaceae strains include MR-1, MR4, ME-7, CN-32, and PV-4.

Representative, non-limiting examples of suitable Geobacteraceae species and strains are provided in Table 2 below. As shown in the table, the listed species and strains generally overlap with respect to acceptable conditions of temperature, salinity, and carbon source, and are therefore compatible for growth as mixed cultures. All Geobacteraceae species and strains listed in the table are capable of reduction of both iron and manganese. Additionally, all listed Geobacteraceae species and strains are facultative anaerobes, capable of respiration of oxygen.

TABLE 2

| Bacterial | T | | | Carbon |
|---|---|---|---|---|
| Strain(s) | (° C.) | Salinity | Metals | utilized |
| G. metallireducens GS-15 | 30 | L-M | Fe/Mn | acetate |
| G. sulfurreducens PCA | 10-30 | L-H | Fe/Mn/S | acetate |
| G. uraniireducens | 10-30 | L-M | Fe/Mn/U | acetate |
| G. sulfurreducens KN400 | 10-30 | L-M | Fe/Mn/S | acetate |
| G. hydrogenophilus | 10-30 | L-M | Fe/Mn | acetate |
| G. psychrophilus | 4-20 | L-M | Fe/Mn | acetate |
| G. bremensis | 10-30 | L-M | Fe/Mn | acetate |
| G. bimidjiensis | 10-30 | L-M | Fe/Mn | acetate |
| G. humireducens | 10-30 | L-M | Fe/Mn | acetate |
| G. chapellei | 10-30 | L-M | Fe/Mn | acetate |

Non-limiting examples of particularly preferred Geobacteraceae strains include GS-15 and PCA.

The Geobacteraceae strains are quite different from the Shewanellaceae strains with respect to their growth requirements and preferred carbon source: with few exceptions, most of the described Geobacteraceae strains will not utilize lactate, and all will utilize acetate, making them ideal partners with the acetate-excreting Shewanellaceae in an anoxic metal reducing consortium. In addition, the Geobacteraceae are far more sensitive to oxygen, some being rapidly killed or strongly inhibited by low levels of oxygen. To this end, the ability of the Shewanellaceae strains to respire oxygen and maintain an anaerobic environment is also a valuable trait for any consortium demanding anoxic conditions.

In an exemplary embodiment, the starting materials comprise birnessite and/or todorokite, and the metal-oxide reducing bacteria comprise at least one species selected from Shewanella spp.

In an exemplary embodiment, the starting materials comprise iron oxides, and the metal-oxide reducing bacteria comprise at least one species selected from Shewanella spp. and at least one species selected from Geobacter spp.

In an exemplary embodiment, the bio-extraction stage is carried out at room temperature (i.e., a temperature of from about 20° C. to about 30° C.), and the metal-oxide reducing bacteria comprise at least one species selected from Shewanella spp. and at least one species selected from Geobacter spp.

In an exemplary embodiment, the bio-extraction stage is carried out in an aqueous medium comprising seawater, and the metal-oxide reducing bacteria comprise at least one species selected from Shewanella spp. and at least one species selected from Geobacter spp.

In an exemplary embodiment, the bio-extraction stage is carried out in an aqueous medium comprising lactate, and the metal-oxide reducing bacteria comprise at least one species selected from Shewanella spp.

In an exemplary embodiment, the bio-extraction stage is carried out in an aqueous medium comprising acetate, and the metal-oxide reducing bacteria comprise at least one species selected from Geobacter spp.

In an exemplary embodiment, the comminution and softening the material subcomponents of the preprocessing stage are carried out in an aqueous medium comprising at least one species selected from Shewanella spp. and at least one species selected from Geobacter spp. For example, the comminution subcomponent may be carried out in an aqueous medium comprising at least two species selected from Shewanella spp. and at least one species selected from Geobacter spp.

### Starting Materials

Generally, the starting material may comprise any natural or manufactured material comprising one or more trace metals.

Non-limiting examples of trace metals that may be present in the starting material include lithium, zinc, copper, chromium, nickel, cobalt, vanadium, and molybdenum. Other examples of trace metals that may be present in the starting material include transition metals such as cadmium; as well as rare earth elements such as lanthanum, cerium, neodymium, samarium, europium, terbium, and dysprosium. Additional examples of trace metals that may be present in the starting material include platinum group metals (e.g., iridium, osmium, palladium, platinum, rhodium, and ruthenium). Examples of preferred trace metals include nickel and cobalt.

### Metallic Ores

The starting material may comprise one or more metallic ores. Non-limiting examples of metallic ores include terrestrial sulfur-rich ores, oxides, laterite ores and non-terrestrial ores (e.g. chondrites or asteroids).

Sulfur-rich ores may be utilized as starting materials, for example, nickeliferous pyrrhotite ore. For example, the starting material may comprise pyrrhotite or pentlandite.

Laterite ores that may be utilized as starting materials include, for example, limonite and saprolite. Limonite ores typically have a nickel concentration of from about 1% to about 2%. For example, the starting material may comprise a high-grade limonite ore, a low grade limonite ore, or a combination thereof.

### Clay Minerals

The starting material may comprise one or more iron-rich clay minerals. Non-limiting examples of iron-rich clay minerals include smectite, illite, chlorites, gibbsite, boenite, and diaspore.

### Polymetallic Nodules

Polymetallic nodules are another natural source of trace metals that may be utilized as a starting material. Polymetallic nodules are naturally occurring rock concretions found on the seabed, and typically contain a wide range of trace metals including copper (typically about 1-1.4%), cobalt (typically about 0.2-0.25%), and nickel (typically about 1-1.5%).

Polymetallic nodules typically comprise a manganese oxide content of from about 30% by weight to about 50% by weight. The iron content of polymetallic nodules is highly variable, but in many cases can be 20% by weight or higher.

### Trace Metal-Containing Waste Streams

Many industrial and municipal waste streams contain recoverable amounts of trace metals that make them suitable for use as starting materials for processes and methods provided herein. These include, but are not limited to, batteries, electronic waste, wastewater produced from oil and gas operations, source water from geothermal production, and fly ash.

### Preprocessing Stage

The processes and methods provided herein may comprise a pretreatment or preprocessing stage wherein a starting material is pre-treated prior to the bio-extraction stage. The preprocessing stage may comprise one or more of the following steps or subcomponents: a) comminution, b) softening the starting material, and c) oxidizing the starting material. Each preprocessing step may also be used in combination with another or multiple preprocessing steps.

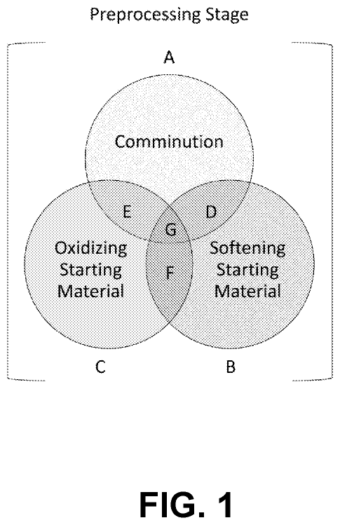

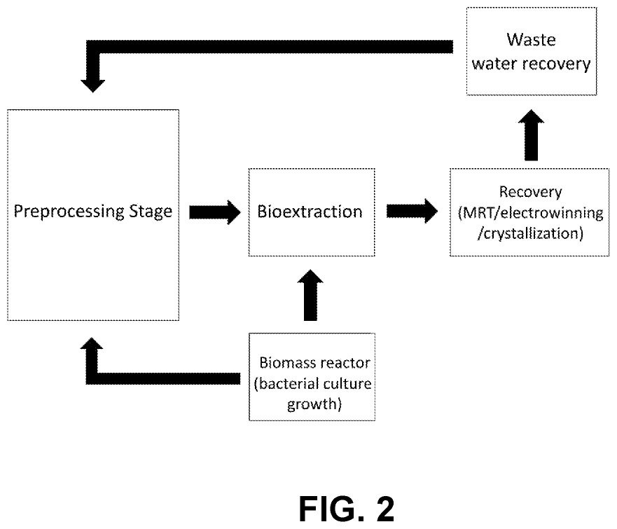

The preprocessing stage may optionally comprise one or more processes for reducing the particle size of the starting material, one or more processes for softening the starting material, one or more processes for oxidizing the starting material, or a combination thereof. Exemplary combinations of these preprocessing subcomponents are shown in FIG. 1. As an illustrative and non-limiting example, the starting material may be first comminuted, then the material softened prior to the bio-extraction stage (combination D in FIG. 1). In this example, the comminution (A in FIG. 1) and softening the material (B in FIG. 1) subcomponents may be combined (D in FIG. 1) to operate separately or together (at the same time) but preferably before the bio-extraction stage (FIG. 2).

As used herein, the term “preprocessed starting material” refers to a starting material that has been subjected to a preprocessing stage as described herein.

### Comminution

The processes and methods provided herein may comprise a comminution step or subcomponent wherein a starting material is comminuted, thereby producing a granular material. The starting material may be crushed, ground, cut, vibrated, or otherwise subjected to methods known in the mineral processing industry for producing a granular material having a smaller average particle size than the starting material.

In preferred embodiments, at least a portion of the starting material is a preprocessed starting material. For example, the starting material entering the comminution step preferably has a mean particle size of less than about 50 mm, less than about 20 mm, less than about 15 mm, less than about 10 mm, less than about 9 mm, less than about 8 mm, less than about 7 mm, less than about 6 mm, less than about 5 mm, less than about 4 mm, less than about 3 mm, less than about 2 mm, or even less than about 1 mm.

In preferred embodiments, at least a portion of the trace metal present inside the starting materials is exposed to the surface of the granular material produced by comminution.

In a preferred embodiment, the comminution comprises a sea water slurry grinding process. The process may be a continuous process or a batch process. The process may utilize grinding media known in the art, including but not limited to ceramic ball grinding media or stainless steel ball grinding media.

The comminution typically produces a granular material having a mean particle size of less than about 1 mm. For example, the granular material exiting the comminution may have a mean particle size of less than about 500 microns, less than about 400 microns, less than about 300 microns, less than about 200 microns, or less than about 100 microns.

The granular material may have a particle size distribution such that at least about 50%, at least about 60%, at least about 70%, at least about 80%, at least about 90%, or at least about 95% of the particles have a diameter of less than about 200 micrometers (μm). For example, the granular material may have a particle size distribution such that at least about 50%, at least about 60%, at least about 70%, at least about 80%, at least about 90%, or at least about 95% of the particles have a diameter of from about 200 micrometers to about 10 micrometers.

Alternatively, when the process comprises a heap leaching step, the granular material may have a particle size distribution such that at least about 50%, at least about 60%, at least about 70%, at least about 80%, at least about 90%, or at least about 95% of the particles have a diameter of less than about ¾″ (19 mm), less than about ½″ (12 mm), or less than about ⅜″ (9.5 mm). Suitable particle sizes may be obtained, for example, by closed circuit crushing.

The preprocessing stage may comprise one or more processes for grinding, crushing, or otherwise reducing the particle size of the starting material. For example, the preprocessing stage may comprise mechanically crushing the starting material.

Equipment suitable for mechanically crushing ores is generally known to those skilled in the art. Non-limiting examples of equipment that may be used to reduce the particle size of the starting material include high specific energy crushers, such as Impact Crushers and High Pressure Grinding Roll (HPGR) crushers, and vibrating high frequency screens. For fine grinding of less than 100 μm, the preferred equipment is vertical mills and high-intensity mills such as vertical HIG mills or horizontal mills such as Isamill.

Typically, the preprocessing stage produces a preprocessed starting material having a mean particle size of less than about 100 mm. For example, the preprocessed starting material may have a mean particle size of less than about 50 mm, less than about 20 mm, less than about 15 mm, less than about 10 mm, less than about 9 mm, less than about 8 mm, less than about 7 mm, less than about 6 mm, less than about 5 mm, less than about 4 mm, less than about 3 mm, less than about 2 mm, or even less than about 1 mm.

### Softening the Starting Material

The preprocessing stage may comprise one or more processes for softening the starting material (e.g., subcomponent B as shown in FIG. 1). This preprocessing subcomponent may be combined with other preprocessing subcomponents described herein.

For example, the preprocessing stage may comprise contacting the starting material with bacteria. The starting material may be at least partially submerged in an aqueous medium comprising one or more species of metal-oxide reducing bacteria. For example, the aqueous medium may comprise at least one species selected from Shewanella spp., and at least one species selected from Geobacter spp., or a combination thereof. The starting material may be submerged in a pretreatment pond or lagoon comprising a population or consortium of bacteria, where it is softened through action of the bacteria. The aqueous medium may comprise seawater, brackish water, or freshwater, and will be inoculated with one or more species of metal-oxide reducing bacteria, which may be selected as described in detail above. Alternatively, the starting material may be submerged within a bioreactor comprising a population or consortium of bacteria. As a further alternative, a crushed starting material may be disposed of in piles or heaps, and then irrigated with an aqueous medium comprising one or more species of metal-oxide reducing bacteria.

When the process comprises a heap leaching step, the preprocessing stage may comprise wetting the ore with solution loaded with high bacteria concentration to a value less than the moisture saturation limit and mixing it in an agglomerating drum, then the moistened material is placed in piles for a period of time.

### Oxidizing the Starting Material

The pretreatment or preprocessing stage may comprise one or more processes for biologically oxidizing the starting material before the bio-extraction process. This pretreatment stage is suitable for ores that are predominantly in the reduced state. A non-limiting example of such material is nickeliferous pyrrhotite, which in addition to nickel, contains reduced sulfur and reduced iron.

For example, the pretreatment stage may comprise of contacting the starting material with neutrophilic (i.e., not acidophilic) metal-oxidizing and/or sulfur-oxidizing bacteria (which may be referred to herein as “nMO/nSOB”). The starting material may be at least partially submerged in an aqueous medium comprising one or more species of nMO/nSOB. The starting material may be submerged within a bioreactor comprising a population or community of nMO/nSOB. Alternatively, the starting material may be submerged in a pretreatment pond or lagoon comprising a population or community of bacteria, where it is oxidized through action of nMO/nSOB.

As an illustrative example, the pretreatment stage may utilize one or more species of nMO/nSOB.

Non-limiting examples of neutrophilic sulfur-oxidizing bacteria include the taxonomic order Thiotrichales. For example, bacteria in the order Thiotrichales have the ability to partially oxidize sulfur to elemental sulfur at neutral pH. Members of the Thiotrichales utilize sulfur as a source of energy. The sulfur in the starting material is turned to elemental sulfur and/or other sulfur intermediates, exposing and concentrating more of the iron, manganese, and/or trace metals in the starting material.

Non-limiting examples of neutrophilic metal-oxidizing bacteria include the taxonomic family of Gallionellaceae. For example, bacteria in the family Gallionellaceae have the ability to oxidize iron at neutral pH. Members of the Gallionellaceae utilize iron as a source of energy. The iron in the starting material is turned to rust, which can then be reduced in the subsequent bio-extraction stage.

Representative, non-limiting examples of suitable freshwater and marine nMO/nSOB genera for this pretreatment stage are provided in Table 3 below.

TABLE 3

| | | | nMO or | Freshwater |
|---|---|---|---|---|
| Order | Family | Genus | nSOB | or Marine |
| Thiotrichales | Piscirickettsiaceae | Hydrogenovibrio | nMO/nSOB | M |
| Thiotrichales | Piscirickettsiaceae | Thiomicrospira | nSOB | M |
| Thiotrichales | Piscirickettsiaceae | Thiomicrorhabdus | nSOB | M |
| Thiotrichales | Beggiatoaceeae | Beggiatoa | nSOB | F/M |
| Thiotrichales | Piscirickettsiaceae | Sulfurivirga | nSOB | M |
| Thiotrichales | Piscirickettsiaceae | Thioalkalimicrobium | nSOB | M |
| Thiotrichales | Thiothricaceae | Thiothrix | nSOB | F/M |
| Mariprofundales | Mariprofundaceae | Mariprofundus | nMO | M |
| N/A | N/A | Ghiorsea | nMO | M |
| Gallionellales | Gallionellaceae | Gallionella | nMO | F |
| Gallionellales | Gallionellaceae | Ferriphaselus | nMO | F |
| Gallionellales | Gallionellaceae | Ferrigenium | nMO | F |
| Gallionellales | Gallionellaceae | Sideroxydans | nMO/nSOB | F |
| Desulfobacterales | Desulfobulbaceae | Desulfobulbus | nSOB | F/M |

### Bio-Extraction Stage

The processes and methods provided herein may comprise a bio-extraction stage wherein a granular material comprising one or more trace metals is contacted with one or more species of metal-oxide reducing bacteria. The metal-oxide reducing bacteria convert at least a portion of the metal oxide to a water-soluble metal salt, thereby releasing at least a portion of the trace metal into the aqueous medium.

The granular material may be produced, for example, by subjecting a starting material to comminution as described above. It is particularly desirable that the particle size distribution of the granular material falls substantially below about 200 microns, as generally described above. Laboratory results have indicated that particles of manganese oxide, iron oxide, or a combination thereof having a diameter of about 200 microns or less will be rapidly and completely electrochemically reduced when subjected to a bio-extraction stage as provided herein.

In general, the granular material will have the same composition as the starting materials as described above.

The granular material may comprise one or more metal oxides. For example, the granular material may comprise one or more iron oxide compounds, one or more manganese oxide compounds, or a combination thereof. When the granular material is contacted with one or more species of metal-oxide reducing bacteria, at least a portion of the metal oxides present in the granular material are electrochemically reduced, thereby producing one or more metal oxide salts. In preferred embodiments, the produced metal oxide salts are water soluble. For example, the metal oxide salt may be a metal chloride salt.

For example, the granular material may comprise at least one iron oxide compound. As used herein, the term “iron oxide” refers to any compound of the form FeₓO_{y}H_{z} wherein x is greater than or equal to one, y is greater than or equal to one, and z is greater than or equal to zero. Non-limiting examples of iron oxides that may be present in the granular material include FeOOH, Fe(OH)₃, Fe₂O₃, FeO, FeO₂, Fe₃O₄, Fe₄O₅, Fe₅O₆, Fe₅O₇, Fe₂₅O₃₂, and Fe₁₃O₁₉. Non-limiting examples of minerals that comprise iron oxides include wüstite, magnetite, hematite, and maghemite.

When the granular material is contacted with the one or more species of metal oxide reducing bacteria, at least a portion of the iron oxide is electrochemically reduced to form an iron salt, which is preferably a water-soluble iron salt. As a non-limiting example, at least a portion of the iron oxide may be converted to iron chloride.

In preferred embodiments, at least about 50% by weight of the iron oxide present in the granular material is converted to a water-soluble iron salt (for example, iron chloride). For example, at least about 55%, at least about 60%, at least about 65%, at least about 70%, at least about 75%, at least about 80%, at least about 85%, at least about 90%, or at least about 95% by weight of the iron oxide present in the granular material may be converted to a water-soluble iron salt.

The granular material may comprise at least one manganese oxide compound. As used herein, the term “manganese oxide” refers to any compound of the form MnₓO_{y}H_{z} wherein x is greater than or equal to one, y is greater than or equal to one, and z is greater than or equal to zero. Non-limiting examples of manganese oxides that may be present in the granular material include MnO, Mn₃O₄, Mn₂O₃, MnO₂, MnO₃, Mn₂O₇, Mn₅O₈, Mn₇O₁₂, and Mn₇O₁₃. Non-limiting examples of minerals that comprise manganese oxides include birnessite, hausmannite, manganite, manganosite, psilomelane, pyrolusite, bixbyite, jacobsite, columbite, tantalite, coltan, galaxite, and todorokite. For example, the predominant manganese oxides in polymetallic nodules are a combination of minerals with same general formula (MnO₂): δ-MnO₂, birnessite, and todorokite.

When the granular material is contacted with the one or more species of metal oxide reducing bacteria, at least a portion of the manganese oxide is electrochemically reduced to form a manganese salt, which is preferably a water-soluble manganese salt. As a non-limiting example, at least a portion of the manganese oxide may be converted to manganese chloride.

In preferred embodiments, at least about 50% by weight of the manganese oxide present in the granular material is converted to a water-soluble manganese salt (for example, manganese chloride). For example, at least about 55%, at least about 60%, at least about 65%, at least about 70%, at least about 75%, at least about 80%, at least about 85%, at least about 90%, or at least about 95% by weight of the manganese oxide present in the granular material may be converted to a water-soluble manganese salt.

Laboratory experiments have indicated that up to 95% of the MnO₂ will be converted to soluble MnCl₂ by Shewanella strains, with a similar yield being achieved for the iron oxides by Geobacter strains, yielding soluble FeCl₃.

In preferred embodiments, at least about 10% by weight of the trace metal present in the granular material is released into the aqueous medium. For example, at least about 20%, at least about 30%, at least about 40%, at least about 50%, at least about 60%, at least about 70%, at least about 80%, at least about 90%, or at least about 95% by weight of the trace metal present in the granular material may be released into the aqueous medium.

In some embodiments, the bio-extraction stage comprises a continuous or semi continuous process wherein (a) an aqueous input stream comprising one or more species of metal-oxide reducing bacteria and (b) a granular material are fed into a reaction vessel, thereby producing (c) an aqueous output stream comprising suspended solids, dissolved metals, and bacteria.

In preferred embodiments, at least a portion of the aqueous input stream is derived from a biomass production and separation stage as described below. The aqueous input stream may further comprise seawater, brackish water, or freshwater.

The output from the bio-extraction stage may be an aqueous stream containing suspended solids, dissolved metals, and bacteria. This aqueous output stream may be referred to herein as an “aqueous metallic slurry.” In general, the aqueous metallic slurry will have the same overall composition as the granular material entering the bio-extraction stage.

The bio-extraction stage should be carried out in a reaction vessel comprising an aqueous medium suitable for the activity of the selected bacteria. In preferred embodiments, the aqueous medium is maintained at a neutral or near-neutral pH. For example, the pH of the aqueous medium is typically no less than about 5, no less than about 5.5, no less than about 6, or no less than about 6.5. Likewise, the pH of the aqueous medium is typically no greater than about 9, no greater than about 8.5, no greater than about 8, or no greater than about 7.5.

The reaction vessel should have a geometry suitable for contacting the bacterial cells with the granular material. Additionally, the reaction vessel should be capable of maintaining an efficient flow rate to keep the bacterial cells well mixed throughout the volume containing the granular material. In preferred embodiments, the reaction vessel is an upflow reactor (also known in the art as a UASB reactor).

Alternatively, the bio-extraction stage may comprise a heap leach process. In a heap leach process, the granular material is arranged in a heap comprising one or more layers. Typically, the heap may be from about 5 meters to about 8 meters high.

An aqueous input stream may be applied to the heap using methods known in the art. For example, the aqueous input stream may be applied to the heap using sprinklers. The sprinklers may apply the aqueous input stream at a flow rate, for example, of between about 5 L/h/m² and about 20 L/h/m², depending on the hydraulic transmissibility of the stockpiled ore.

In a heap leach process, the bio-extraction reaction (i.e., where metal-oxide reducing bacteria convert at least a portion of the metal oxide to a water-soluble metal salt) occurs at the thin contact boundary layer of the aqueous input stream solution and the granular material. This reaction produces an aqueous output stream, or an “aqueous metallic slurry,” which flows to the bottom of the heap. Methods for recovering the aqueous metallic slurry from the bottom of the heap are generally known in the art. For example, a waterproof covering may be placed beneath the heap, which collects the aqueous metal slurry.

### Biomass Generation Stage

The processes and methods provided herein may comprise a biomass generation stage, wherein a bioreactor is used to grow and/or maintain a population or community of one or more species of metal-oxide reducing bacteria.

The biomass generation stage may comprise a biomass reactor suitable for growing and maintaining a population or community of bacteria. The biomass reactor may comprise means for maintaining the temperature, pH, salinity, or other aspects of the aqueous medium within the reactor as needed to support the growth of the desired bacterial population/community.

In some embodiments, the biomass generation stage comprises a continuous or semi-continuous process wherein (a) an aqueous input stream is fed into a biomass reactor, thereby producing (b) an aqueous output stream comprising the one or more species of metal oxide reducing bacteria. The aqueous input stream may comprise seawater, brackish water, or freshwater.

The aqueous input stream may optionally comprise a bacterial growth substrate. The selection of a suitable bacterial growth substrate will depend on the particular species of metal-oxide reducing bacteria present within the biomass reactor. Non-limiting examples of suitable bacterial growth substrates include sodium lactate and sodium acetate.

In some embodiments, the aqueous input stream comprises at least a portion of the aqueous return stream produced by the biomass separation stage, as described generally below. For example, the biomass generation stage may comprise a means for retaining the biomass within the reaction vessel. For example, the biomass generation stage may comprise a filter or membrane. The membrane may be, for example, a hollow fiber membrane.

### Biomass Separation Stage

The processes and methods provided herein may comprise a biomass separation stage that comprises separating and retaining at least a portion of the metal-oxide reducing bacteria present in the aqueous metallic slurry produced by the bio-extraction stage.

The biomass separation stage comprises a means for separating metal-oxide reducing bacteria from the aqueous metallic slurry, while allowing dissolved metals to pass through to the recovery stage. The separation stage may further comprise means for controlling the temperature, pH, total dissolved solids (TDS), mixing and flow rates.

In some embodiments, the dissolved trace metals in the slurry from the bio extraction stage will be separated from the metal-oxide reducing bacteria using methods for removing the trace metals including, but not limited to absorption, chemical modification, and filtration. The filtration may include forward osmosis membrane technology.

In some embodiments, the metal-oxide reducing bacteria are retained from the dissolved trace metals in the slurry from the bio-extraction stage by filtration using a microfilter combined in a semi-closed loop configuration to the biomass reactor.

In some embodiments, the metal-oxide reducing bacteria are retained from the dissolved trace metals in the slurry from the bio-extraction stage by membrane filtration. The membrane may be, for example, a hollow fiber membrane.

### Recovery Stage

The processes and methods provided herein may comprise a recovery stage wherein trace metals are separated and recovered from an aqueous metallic slurry. The aqueous metallic slurry may comprise the output of a bio-extraction stage and/or a biomass production and separation stage as described above.

Means for separating dissolved trace metals from an aqueous solution are generally known to those skilled in the art. Non-limiting examples of suitable methods and techniques include solvent extraction (“SX”), ion exchange (“IX”), chelating ion exchange resins (“CIXR”), molecular recognition technology (“MRT”), electro-winning (“EW”), and crystallization as sulfate or carbonate, among others.

After the separation of the metals, the spent solutions may be conditioned to return to the bio-extraction stage, or to the biomass process. Optionally, water losses due to evaporation in the EW or hydration of the crystallization products may be balanced through the addition of make-up water.

In some embodiments, the aqueous metallic slurry comprises iron, manganese, or a combination thereof in a concentration of about 100 μM, about 200 μM, about 300 μM, about 400 μM, about 500 μM, about 600 μM, about 700 μM, about 800 μM, about 900 μM, about 1 mM, about 2 mM, about 3 mM, about 4 mM, or about 5 mM or more.

In some embodiments, the aqueous metallic slurry comprises one or more trace metals in a concentration of about 1 μM, about 2 μM, about 5 μM, about 10 μM, about °μM, about 30 μM, about 40 μM, about 50 μM, or about 100 μM or more.

### Conversion of Iron and Manganese to Insoluble Minerals

The processes and methods provided herein may comprise a mineral conversion stage wherein recovered iron and manganese are converted to insoluble minerals that are suitable for return to the environment.

For example, the recovered iron and manganese may be converted to iron and manganese oxides. The iron oxide conversion may be achieved after a conventional separation stage (e.g. Molecular Recovery Technology (MRT)), by running the recovered solution through an aerobic cycle that will rapidly (within 1-2 hours at most) convert the iron to an insoluble iron hydroxide. A similar treatment can be used for the manganese, but will require raising the pH to 9 or above to speed up the precipitation.

Alternatively, both metals could be converted to their respective carbonates (MnCO₃ (rhodochrosite) and FeCO₃ (siderite)), both of which are insoluble. The latter method may be preferred in some cases, as it would yield carbon credits for disposing of solid CO₂.

### Trace Metals

Trace metals, obtained using the methods described above, are also within the scope of the present disclosure.

Non-limiting examples of trace metals that may be recovered using the methods provided herein include lithium, zinc, copper, chromium, nickel, cobalt, vanadium, and molybdenum. Other examples of trace metals that may be recovered using the methods provided herein include transition metals such as cadmium; as well as rare earth elements such as lanthanum, cerium, neodymium, samarium, europium, terbium, and dysprosium. Additional examples of trace metals that may be recovered using the methods provided herein include platinum group metals (e.g., iridium, osmium, palladium, platinum, rhodium, and ruthenium). Examples of preferred trace metals include nickel and cobalt.

### EXAMPLES

The following non-limiting examples are provided to further illustrate the present disclosure.

### Example 1: Preparation of Bacterial Inoculum

A bacterial inoculum is grown for a few hours until it reaches a cell density of 10⁹ cells per ml or higher. The initial growth medium is a rich-medium containing tryptone and yeast extract. The cells are grown aerobically in shake flasks. After the growth reaches the proper density, the shaking is stopped, and the cells rapidly became oxygen limited, and began to synthesize the enzymes needed for anaerobic metabolism (e.g., metal reduction). These cells are then added to a suspension of metal oxide (either iron or manganese) to a density of 10⁷ cells per ml, and sodium lactate is added at a molarity sufficiently high for the cells to respire all of the metal oxide. As the reaction proceeds, lactate is consumed by the bacteria, electrons are extracted and used to reduce the metal oxide to soluble salts (MnCl₂ or FeCl₂). Soluble metals and lactate are measured in order to determine the stoichiometry of the process for each bacterial strain or consortium that is tested.

### Example 2: Bio-Extraction of Iron-Bearing Mineral Ores and Manganese Nodules

Initial experiments to determine rates of iron and/or manganese reduction in iron-bearing mineral ores and manganese nodules were conducted using the following methodology.

Iron-bearing mineral ores and manganese nodules were crushed and ground using different, clean, ceramic mortars and pestles. This material was sieved to sizes<100 um. The material was autoclaved to control for potential contamination. Shewanella loihica PV-4 (hereafter PV-4) was grown initially in LB broth medium at pH 7 (per liter: 10 g tryptone, 5 g yeast extract, 5 g sodium chloride) overnight at 18° C. in a shaker platform to achieve cell density of 10⁹ cells/mL. The culture was centrifuged at 5000 rpm for 30 minutes to pellet the cells, which were resuspended in M1 minimal medium (per liter: 15.1 g PIPES buffer, 3.4 g sodium hydroxide, 1.5 g ammonium chloride, 0.1 g potassium chloride, 0.6 g sodium phosphate monobasic monohydrate; with vitamins, trace minerals, and amino acid solutions as supplements; either 18 mM lactate as electron donor or 18 mM lactate+15 mM glucose as electron donors) to yield an optical density of >2.00 at 600 nm wavelength. This concentrated mixture was used as the inoculum for the experiments with iron ore/nodule material in M1 medium. The inoculum to total volume of medium ratio was 1:100. The ratio of amount of nodule material to total volume of medium was measured as 0.5 and 5 g/L. The cultures were either shaken at 150 rpm or incubated statically. PV-4 was tested anaerobically and microaerobically. Anaerobic conditions were achieved by sparging the medium with pure N₂ (g) for at least 30 minutes. The medium is autoclaved in an atmosphere of N₂ (g) for 30 minutes. The medium is cooled-down to room temperature. At this point, ground, sieved, sterile iron ore/nodule material was mixed with 1 mL of medium and subsequently introduced into the culture vessel using a syringe. The medium was sparged with N₂ (g) for an additional 5 minutes. For microaerobic conditions, there was no further sparging of the medium with N₂ (g) after the medium was autoclaved.

Dissolved manganese concentrations were measured by the formaldoxime method (Sigma Aldrich Spectroquant Manganese Test; absorbance measured at 450 nm). For crushed manganese nodule, the dissolved manganese increased linearly with increasing amounts of nodule added in the presence of PV-4. For example, the concentration of dissolved manganese was >10 times more concentrated in the 5 g/L experiment versus the 0.5 g/L experiment. The dissolved manganese was 24% and 29% for 0.5 and 5 g/L, respectively. The total manganese content was measured after treatment with 6.25 N hydroxylamine and 0.5 N HCl. These results were surprising and encouraging because it indicates that the amounts of manganese nodules that can react in the bio-extraction step can be increased beyond 5 g per L of medium, expecting a linear correlation to dissolved manganese (and other associated metals).

Dissolved (no pretreatment), adsorbed Fe (treated with 0.5 N HCl for 1 hour), and extractable Fe (defined as the total Fe dissolved with 6.25 N hydroxylamine and 0.5 N HCl) were measured by the ferrozine method (2 mM ferrozine iron reagent in 50 mM HEPES buffer; absorbance measured at 562 nm). For the 0.5 g/L iron-bearing tailing sample 1 experiment (higher-grade), the dissolved Fe was 1% and 4% within 24 hrs and 96 hrs, respectively. For the 5 g/L sample 1 experiment, the dissolved Fe was <1% and <1% within 24 hrs and 96 hrs, respectively. For the 0.5 g/L iron-bearing tailing sample 2 (lower-grade), the dissolved Fe was 9% and 20% within 24 hrs and 96 hrs, respectively. For the 5 g/L sample 2 experiment, the dissolved Fe was 1% and 2% within 24 hrs and 96 hrs, respectively. Taking into account the combined dissolved and adsorbed Fe (biologically reduced Fe that subsequently binds to mineral) for the 0.5 g/L experiments, the level of released Fe at 96 hrs was 3% and 41% for sample 1 and 2, respectively, highlighting the more reactive nature of the tailing sample 2 which bound more of the dissolved Fe (adsorbed Fe). For the 5 g/L experiments at 96 hrs, the combined dissolved and adsorbed Fe was 6% and 7% for sample 1 and 2, respectively. Overall, these initial experiments show substantial amounts of Fe being reduced by PV-4, especially with the low-grade tailing sample 2 (>40% extractable Fe is biologically reduced).

Having described the disclosure in detail, it will be apparent that modifications and variations are possible without departing from the scope of the claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

1. A method for the extraction of a trace metal from a granular material comprising a metal oxide, the method comprising:
contacting the granular material with at least one species of metal-oxide reducing bacteria in an aqueous medium, thereby (1) converting at least a portion of the metal oxide to a water soluble metal salt, and (2) releasing at least a portion of the trace metal into the aqueous medium; and
recovering at least a portion of the trace metal from the aqueous medium.

2. The method of claim 1 wherein the at least one species of metal-oxide reducing bacteria comprises at least one species selected from Shewanella spp., at least one species selected from Geobacter spp., or a combination thereof.

3. The method of claim 2 wherein the at least one species of metal-oxide reducing bacteria comprises at least one species selected from Shewanella spp.

4. The method of claim 3 wherein the at least one species of metal-oxide reducing bacteria comprises at least one species selected from the group consisting of:
S. oneidensis MR-1,
S. amazonensis SB2B,
S. baltica OS185,
S. putrefaciens CN-32,
S. loihica PV-4,
S. sp. MR-4,
S. sp. MR-7,
S. sp. W3-18-1,
S. sp. ANA-3, and
S. violaceae DSS12.

5. The method of claim 2 wherein the at least one species of metal-oxide reducing bacteria comprises at least one species selected from Geobacter spp.

6. The method of claim 4 wherein the at least one species of metal-oxide reducing bacteria comprises at least one species selected from the group consisting of:
G. metallireducens GS-15,
G. sulfurreducens PCA,
G. uraniireducens,
G. sulfurreducens KN400,
G. hydrogenophilus,
G. psychrophilus,
G. bremensis,
G. bimidjiensis,
G. humireducens, and
G. chapellei.

7. The method of claim 2 wherein the at least one species of metal-oxide reducing bacteria comprises at least one species selected from Shewanella spp. and at least one species selected from Geobacter spp.

8. The method of claim 1 wherein the granular material comprises a metal oxide selected from the group consisting of iron oxides and manganese oxides.

9. The method of claim 8 wherein the granular material comprises an iron oxide.

10-12: (canceled)

13. The method of claim 9 wherein at least about 50% by weight of the iron oxide present in the granular material is converted to a water-soluble iron salt.

14. The method of claim 8 wherein the granular material comprises a manganese oxide.

15-16: (canceled)

17. The method of claim 14 wherein at least about 50% by weight of the manganese oxide present in the granular material is converted to a water-soluble manganese salt.

18. The method of claim 1 wherein the water-soluble metal salt is selected from the group consisting of iron chloride and manganese chloride.

19. The method of claim 1 wherein the trace metal is selected from the group consisting of lithium, zinc, copper, chromium, nickel, cobalt, vanadium, and molybdenum.

20: (canceled)

21. The method of claim 1 wherein at least about 10% by weight of the trace metal present in the granular material is released into the aqueous medium.

22-27: (canceled)

27. The method of claim 1 further comprising a preprocessing stage wherein a starting material is subjected to one or more steps selected from (a) comminution, (b) softening the starting material, and (c) oxidizing the starting material,
thereby producing a granular material comprising a metal oxide.

28-52: (canceled)

53. A method for the extraction of a trace metal from a granular material comprising a metal sulfide, the method comprising:
(1) contacting the granular material with at least one species of bacteria selected from the group consisting of neutrophilic metal-oxidizing bacteria and sulfur-oxidizing bacteria in an aqueous medium, thereby converting at least a portion of said metal sulfide to a metal oxide;
(2) subsequently contacting the granular material with at least one species of metal-oxide reducing bacteria in an aqueous medium, thereby (a) converting at least a portion of the metal sulfide to a water soluble metal salt, and (b) releasing at least a portion of the trace metal into the aqueous medium; and
(3) recovering at least a portion of the trace metal from the aqueous medium.

54. The method of claim 53 wherein the granular material is contacted with at least one species of neutrophilic sulfur-oxidizing bacteria selected from the group consisting of Thiotrichales spp.

55. The method of claim 53 wherein the granular material is contacted with at least one species of neutrophilic metal-oxidizing bacteria selected from the group consisting of Gallionellaceae spp.

56-57: (canceled)

58. A method for comminuting a starting material comprising a metal oxide, the method comprising:
at least partially submerging the starting material in an aqueous comminution medium comprising at least one species of metal-oxide reducing bacteria; and
mechanically crushing or vibrating the starting material;
thereby producing a granular material having a smaller mean particle size than the starting material.

59. The method of claim 58 wherein the aqueous comminution medium comprises at least one species selected from Shewanella spp., at least one species selected from Geobacter spp., or a combination thereof.

60. The method of claim 59 wherein the at least one species of metal-oxide reducing bacteria comprises at least one species selected from Shewanella spp.

61. The method of claim 60 wherein the at least one species of metal-oxide reducing bacteria comprises at least one species selected from the group consisting of:
S. oneidensis MR-1,
S. amazonensis SB2B,
S. baltica O S185,
S. putrefaciens CN-32,
S. loihica PV-4,
S. sp. MR-4,
S. sp. MR-7,
S. sp. W3-18-1,
S. sp. ANA-3, and
S. violaceae DSS12.

62. The method of claim 59 wherein the at least one species of metal-oxide reducing bacteria comprises at least one species selected from Geobacter spp.

63. The method of claim 62 wherein the at least one species of metal-oxide reducing bacteria comprises at least one species selected from the group consisting of:
G. metallireducens GS-15,
G. sulfurreducens PCA,
G. uraniireducens,
G. sulfurreducens KN400,
G. hydrogenophilus,
G. psychrophilus,
G. bremensis,
G. bimidjiensis,
G. humireducens, and
G. chapellei.

64. The method of claim 63 wherein the at least one species of metal-oxide reducing bacteria comprises at least one species selected from Shewanella spp. and at least one species selected from Geobacter spp.

65-67: (canceled)

68. The method of claim 58 wherein the starting material comprises a metal oxide selected from the group consisting of iron oxides and manganese oxides.

69-78: (canceled)
